(19) United States
(12) Patent Application Publication
(10) Pub. No.: US 2007/0031444 A1
(43) Pub. Date: Feb. 8, 2007

(54) COMPOUNDS TARGETED TO CELLULAR LOCATIONS

(75) Inventors: Pamela Jane Elizabeth Rowling, Essex (GB); Geoffrey Paul Smith, Essex (GB); Simon Hugh Ridley, Essex (GB)

Correspondence Address: PROSKAUER ROSE LLP, 1001 PENNSYLVANIA AVE, N.W.,, SUITE 400 SOUTH, WASHINGTON, DC, 20004, US

(73) Assignee: ADPROTECH LIMITED, Essex (GB)

(21) Appl. No.: 11/427,112

(22) Filed: Jun. 28, 2006

Related U.S. Application Data
(63) Continuation of application No. 10/332,047, filed on Apr. 2, 2003, now abandoned.

(30) Foreign Application Priority Data
Jul. 7, 2000 (GB) GB0016811.2

(51) Int. Cl. A61K 39/00; C07H 21/04; C12P 21/06; C07K 14/775; C07K 14/74; C07K 14/705

(52) U.S. Cl. 424/185.1; 435/69.1; 435/320.1; 435/325; 530/359; 530/350; 536/23.5

(57) ABSTRACT

A soluble derivative of a soluble polypeptide, which comprises two or more heterologous membrane binding elements with low membrane affinity covalently associated with the polypeptide, the elements being soluble in aqueous solution, and the elements being capable of interacting, independently and with themodynamic additivity, with components of cellular or artificial membranes exposed to extracellular fluids, characterized in that the membrane binding elements target lipid raft components of the membrane and bind to the lipid rafts to localize the polypeptide at the lipid rafts.

This invention related to entities that bind to lipid rafts on cells and methods for their productions and use.

The plasma membrane of a cell forms physical barrier that maintains the integrity of the cell and encapsulates the cytoplasm in which essential functions are carried out. The prevailing view of the structure of the cell membrane was described as the fluid mosaic model (Singer and Nicolson, Science 175, 720, 1972) that suggested that the essential structural repeating unit is the phospholipid molecule in a bilayer arrangement with a thickness of about 500 nm. In this model, membrane proteins are ‘dissolved’ in the bilayer and both lipids and proteins are distributed randomly in the membrane. However, it is now clear that different lipid species are distributed non-randomly in the membrane. However, it is now clear that different lipid species are distributed non-randomly over the exoplasmic and endoplasmic leaflets of the membrane (van Meer, Annu. Rev. Cell Biol. 5, 247, 1989). In addition, lipids and proteins in the membrane are also organised in the lateral dimension into microdomains or so-called ‘lipid rafts’ (Simons and Ikonen, Nature 387, 569, 1997).

The principal building blocks of cell membranes are phospholipids. These molecules are esters of glycerol comprising two fatty acyl residues (non-polar tails) and a single phosphate ester substituent (polar head group). Despite their overall similarity, natural phospholipids exhibit subtle differences in their fatty acid composition, degree of acyl chain unsaturation and in the type of polar head group (Dowhan, Ann Rev Biochem. 66, 199, 1997). These differences can produce significant variations in the physical properties of membranes, the location of the phospholipids in the bilayer and in their biological activity. Phosphatidylcholine is distributed equally between the leaflets; by contrast, virtually all of the phosphatidylserine and most of the phosphatidylethanolamine and phosphatidylinositol resides on the cytoplasmic leaflet.

Sphingolipids are largely confined to the outer leaflet of the membrane. Sphingolipids differ from phospholipids because the structural backbone of these molecules is the lipophilic amino-dialcohol sphingosine, rather than glycerol. Sphingolipids include ceramides, sphingomyelins, and glycosphngolipids (glycosylceramides and gagliosides). Glycosphingolipids occur in the plasma membrane of all eukaryotic cells and interact at the cell surface with toxins, viruses and bacteria, as well as with receptors and enzymes and are involved in cell-type-specific cell adhesion processes. Gangliosides have complex oligosaccharide head groups containing at least one sialic acid residue in place of the single galactose or glucose residue of cerebrosides.

One further important constituent of biological membranes of mammals is cholesterol. Cholesterol is present on both leaflets of the membrane and intercalates among the fatty acyl chains, with its hydroxyl group oriented towards the aqueous surface and the aliphatic chain aligned parallel to the acyl chains in the centre of the bilayer. The presence of cholesterol in membranes has a significant effect of the physical properties of the membrane by restricting the freedom of movement of other membrane lipid components and decreasing the fluidity of the membrane. It is thought that the preferential packing of sphingolipids and cholesterol organizes the lipids into a liquid-ordered phase domain thus forming a so-called lipid raft (Rietveld and Simons, Biochem. Biophys. Acta 1376, 467, 1998; Simons and Ikonen, Nature 387, 569, 1997).

Sphingolipid-cholesterol rafts are insoluble in the detergent Triton X-100 at 4 degrees Celsius. Extraction under these conditions leads to the isolation of a membrane fraction termed a detergent-insoluble glycolipid-enriched complexes (DIGs). DIGs are thought to contain the remnants of the cellular raft domains that are aggregated together (Brown and Rose, Cell 68, 533, 1992; Kurzchalia et al., Trends Cell Biol. 5, 187, 1995; Parton and Simons, Science 269, 1398, 1995). Several membrane proteins are specifically enriched in the DIG fraction and are considered to be raft proteins. The characterisation of proteins in DIGs has shown that proteins can selectively be included or excluded from these microdomains. Proteins bound to rafts include a class of proteins that are anchored to the exoplasmic leaflet of the membrane via a glycosylphosphatidylinositol (GPI) moiety (Brown and Rose, Cell 68, 533, 1992) that contains two (usually) saturated fatty acyl chains. The association of GPI-anchored proteins with rafts is dependent on the length of acyl and alkyl chain composition (Benting et al., FEBS Lett. 462, 47, 1999). Some cytoplasmic proteins are also found in the DIG fraction and are thus thought to be associated to raft domains via the cytoplasmic leaflet of the lipid bilayer. These include several signaling molecules such as G-alpha subunits of heterotrimeric G proteins or the doubly acylated Src-family kinases (Casey, Science 268, 221, 1995). Investigation of the association of proteins with lipid rafts has shown that extraction of cholesterol by saponin abolishes the association with the DIG fraction as predicted from the involvement of cholesterol in the formation of lipid rafts (Cemeus et al., J. Biol. Chem. 268, 3150, 1993; Hanada et al., J. Biol. Chem. 270, 6254, 1995; Scheiffele et al., EMBO J. 16, 5501, 1997).

The role of lipid rafts in cellular function is not fully understood. By studying the proteins and lipids that are found in DIGs several functions have been eluciadated. Several signalling molecules partition into DIGs (Parton and Simons, Science 269, 1398, 1995; Anderson, Proc. Natl. Acad. Sci (USA), 90, 10909, 1993; Lisanti et al., Trends Cell Biol., 4, 231, 1994) such as trimeric G proteins (Li et al., J. Biol. Chem., 270, 15693, 1995), src-family kinases (Casey, Science 268, 221, 1995) and Ras (Song et al., J. Biol. Chem., 271, 9690, 1996; Mineo et al., J. Biol. Chem., 271, 11930, 1996). Lipids involved in signal transduction have also been localised in DIGs, including phosphoinositides (hope and Pike, Mol. Biol. Cell, 7, 843, 1996). Furthermore, the clustering of GPI-anchored proteins can activate different signalling pathways depending on the cell type. It is thought that the general function of lipid rafts in signal transduction is to concentrate receptors for interaction with ligands and effector proteins or lipids on both sides of the membrane and by modulating the association of proteins with rafts (Nykjaer et al., J. Biol. Chem 269, 25668, 1994; Field et al., Proc. Natl. Acad. Sci. (USA) 92, 9201, 1995), to also help to ensure specificity and fidelity. In a further function, lipid rafts organise the transport of molecules from the endoplasmic reticulum to the cell surface (Simons and Ikonen, Nature 387, 569, 1997).

Lipid rafts have also been characterised in living cells. The structure of lipid rafts has been studied by comparing the patching behaviour of different membrane proteins and lipids as detected by immunofluorescence microscopy (Harder et al., J. Cell Biol. 141, 929, 1998). In this procedure, the proteins or lipids that are found in DIGs were crosslinked with fluorescently-labelled antibodies and/or chloera toxin. The membrane components formed patches on the cell surface. Patches formed by DIG-associated proteins, such as the GPI-anchored protein placental alkaline phosphotase, coincided with patches formed by the raft lipid ganglioside GM1 which was detected with the fluorescently-labelled B-subunit of cholera toxin. By contrast, the patches that were formed by proteins that were not DIG-associated were segregated from the patches of GPI-linked protein or GM1. Many other proteins have been charaterised as raft-associated by this procedure (reviewed in Brown and London, Annu. Rev. Cell Dev. Biol. 14, 111, 1998).

The organisation of proteins in cholesterol-dependent domains has also been analysed in living cells by fluorescence resonance energy transfer (FRET) microscopy between GPI-anchored proteins (Varma and Mayor, Nature 394, 798, 1998) or by chemically crosslinking GPI-anchored proteins in cholesterol-dependent domains has also been analysed in living cells by fluorescence resonance energy transfer (FRET) microscopy between GPI-anchored proteins on the cell surface (Friedrichson and Kurzchalia, Nature 394, 802, 1998). These studies showed that the GPI-anchored proteins cluster in membrane microdomains that are smaller than those seen after detergent extraction, and need cholesterol to be maintained. The exact size or protein and lipid composition of rafts in living cells is not fully understood.

The aforementioned description of lipid rafts describes the current state-of-the-art which has focused on the visualization of lipid rafts and the characterization of components that make up these domains. Hitherto, the components of lipid rafts have not been the focus of attention as therapeutic targets. The present invention describes a means for the delivery of compounds to lipid rafts for the purpose of modulating higher intra- or extra-cellular activity for therapeutic benefit. Previous attempts to target compounds to lipid rafts have employed large proteins such as cholera toxin which have a specific affinity for known components of rafts (such as the ganglioside GM-1). However, these approaches are limited in application for therapeutic purposes either because of the molecular complexity and significant immunogenicity of the targeting moiety (Nashar et al., Vaccine 11, 235, 1993; Liljequist et al., J. Immunol. Methods, 210, 125, 1997) or because of problems in the formulation of the lipid raft targeted compounds in aqueous solution.

As an example of the latter, GPI-anchored membrane proteins are not currently used for therapeutic purposes primarily because of difficulties in over-expression, extraction, and handling of the membrane-bound forms. In particular, GPI-anchored forms of proteins are insoluble in aqueous solution in the absence of detergents. Although soluble forms of these GPI-anchored proteins can be produced at a significant scale, their failure to localize in the cell membrane leads to a marked loss in potency. These factors impact the application of human regulators of complement activation as therapeutic complement inhibitors. Specifically, decay accelerating factor (DAF, CD55) and CD59 (Protectin, MACIF) are synthesized as GPI-anchored derivatives that confer protection against complement activation on cells bearing these proteins. However, soluble forms of these proteins which lack the GPI anchor have limited complement inhibitory activity (Moran et al. J. Immunol. 149, 1736, 1992). The GPI-anchored forms of both DAF and CD59 are localized on the cell surface in lipid rafts. Methods that direct compounds to lipid rafts would be applicable to engineered soluble forms of GPI anchored proteins and may enhance the potency of these compounds by directing them to the cellular locations of the native GPI-anchored form. Furthermore, such methods could be applied to other soluble proteins known to interact with components of lipid rafts and could modulate cellular activity by interacting with components of lipid raft signaling pathways. A practical requirement of such derivatives for therapeutic applications is that they should be soluble in aqueous solution in the absence of detergents and that they should be formulatable for pharmaceutical use in humans and animals. One further requirement is that the targeting moiety should have minimal antigenicity.

WO98/02454 describes soluble derivatives of soluble polypeptides, which comprise two or more heterologous membrane binding elements with low membrane affinity covalently associated with the polypeptide, the elements being capable of interacting, independently and with thermodynamic additivity, with components of cellular or artificial membranes exposed to extracellular fluids. That invention thus permits the localization of a therapeutic protein at an outer cellular membrane surface. Examples of therapeutic agents which could be modified according to WO98/029454 included but were not restricted to complement regulatory proteins such as CR1 (CD35); DAF (CD55); MCP (CD46); CD 59; Factor H; and C4 binding protein; and hybrids or muteins thereof such as CR1-CD59 (El Feki and Fearon, Mol. Immunol. 33 (supp 1), 57, 1996).

WO 98/02454 also describes a soluble polypeptide that consisted of residues 1-196 of CR1 ([SCR1-3] Cys; Example 6, therein APT154) and was modified by derivatization by a synthetic polypeptide comprising a myristoyl group and a cationic polypeptide sequence (MSWP-1; example 2, therein). The anti-complement activity of the product ([SCR1-3]-Cys-S-S-[MSWP-1]; example 8, therein APT070) was approximately 100-1000-fold more potent than [SCR1-3]-Cys as measured by the classical pathway-mediated haemolysis of sheep erythrocytes.

We have now found that the combinatorial membrane binding element approach that was first described in WO98/02454 can be applied to localize compounds to lipid rafts. The present invention therefore provides a method for the preparation of compounds which, upon derivatization with agents described in WO98/02454 and as herein, localise to lipid rafts. In these compounds, the membrane-binding elements described in WO98/02454 interact selectively with components of lipid rafts including but not restricted to phosphatidylserine, phosphatidyl glycerol, glycosphingolipids, cholesterol, GPI-anchored proteins associated with lipid rafts and other protein components of lipid rafts that may be found normally on the exoplasmic face of the cell. These interactions may be used to modulate the function of lipid rafts either to affect intracellular signaling or to change extracellular functions mediated through the raft domains. Such modification of raft composition may be used to control destructive or pathological processes (such as the action of MAC in complement activation) which are focused upon or mediated through lipid raft regions of cells.

In another embodiment, the invention provides for soluble complement regulatory molecules, including but not restricted to CD59 and DAF, which are targeted to lipid rafts and the signalling pathways that are associated with lipid rafts.

In a further embodiment, the compound is a derivatised antibody, or antibody fragment which can, for example, provide a surrogate receptor localized at a lipid raft to divert a mediator interacting with a lipid raft receptor or which can neutralise a further component of a raft such as a cofactor required for signaling.

In yet a further embodiment, the invention provides for the delivery of a derivatised compound to an intracellular location via targeting to a lipid raft followed by cellular uptake. In a further embodiment, the compound is a derivatised chemical or biological entity that possesses the physical property of fluorescence which enables lipid rafts to be identified and monitored.

In a further embodiment, the compound is a derivatised chemical or biological entity involved in a catalytic process either as an enzyme an enzyme substrate or an enzyme inhibitor.

In a further embodiment, the compound is a derivatised chemical or biological entity that can form a covalent chemical bond with proteins, sugar groups or lipids that are localized in lipid rafts thus permitting the isolation and identification of the raft component. Such compounds include, but are not restricted to entities containing photo-, chemo-, or enzyme-activated crosslinking groups.

WO98/02454 provides a variety of methods for attachment of a soluble polypeptide to the membrane binding elements. As an example, the linkage of these two components was provided by a disulphide bond formed by the reaction of a pyridylthio group on one component with a thiol group on the other component. The thiol group may be a native thiol or one introduced as a protein attachment group (described therein). Alternatively, the protein attachment group can contain a thiol-reactive entity such as the 6-maleimidohexyl group.

Soluble forms of proteins that are normally located in lipid rafts can be produced by either recombinant means or in certain cases purified from a biological source such as human urine or plasma. Such materials may include, but are not restricted to GPI-anchored proteins. These proteins may be treated with a reagent such as 2iminothiolane (described in WO98/02454 and herein). This procedure introduces one or more protein attachment groups into the protein. The modified protein may be separated from excess modifying agents by standard techniques such as dialysis, ultrafiltration, gel filtration and a solvent or salt precipitation. The intermediate material may be stored in frozen solution or lyophilised. The modified protein may be then reacted further with a pyridylthio group that is linked to a membrane binding peptide. In the above process, there can be no guarantee that the chemical modification of the protein by 2-iminothiolane or another thiolating or thiol-reactive agent would produce a polypeptide that retained biological activity. Furthermore, there can be no guarantee that the soluble form of the lipid raft protein which has been chemically linked to entities described in WO98/02454 (which are structurally quite different from GPI anchors would retain the same biological activity as the protein that has been extracted from a lipid raft.

Similar procedures to those described above may also be applied to soluble proteins that do not normally localize in lipid rafts.

In yet another aspect of the invention, the membrane binding peptide may b linked to a terminal cysteine residue which has been introduced into a protein by recombinant methods.

In addition, the polypeptide portion of the derivatives of the invention may be prepared by expression in suitable hosts of modified genes encoding the soluble polypeptide of interest plus one or more peptidic membrane binding elements and optional residues such as cysteine to introduce linking groups to facilitate post transactional derivatization with additional membrane binding elements.

In a further aspect, therefore, the invention provides a process for preparing a derivative according to the invention which process comprises expressing DNA encoding the polypeptide portion of said derivative in a recombinant host cell and recovering the product and thereafter post translationally modifying the polypeptide to chemically introduce membrane binding elements with selectivity for lipid rafts.

In particular, the recombinant aspect of the process may comprise the steps of:
i) preparing a replicable expression vector capable, in a host cell, of expressing a DNA polymer comprising a nucleotide sequence that encodes said polypeptide portion;
ii) transforming a host cell with said vector;
iii) culturing said transformed host cell under condition permitting expression of said DNA polymer to produce said polypeptide; and
iv) recovering said polypeptide.

Where the polypeptide portion is novel, the DNA polymer comprising a nucleotide sequence that encodes the polypeptide portion as well as the polypeptide portion itself and S-derivatives thereof, also form part of the invention. In particular the invention provides a polypeptide portion of a derivative of the invention comprising the soluble peptide linked by a peptide bond to one peptidic membrane binding element and/or including a C-terminal cysteine, and DNA polymers encoding the polypeptide portion.

The recombinant process of the invention may be performed by conventional recombinant techniques such as described in Sambrook et al., Molecular Cloning: A laboratory manual 2nd Edition. Cold Spring Harbor Laboratory Press (1989) and DNA Cloning vols I, II and III (D. M. Glover ed., IRL Press Ltd).

The invention also provides a process for preparing the DNA polymer by the condensation of appropriate mono-, di- or oligomeric nucleotide units.

The preparation may be carried out chemically, enzymatically, or by a combination of the two methods, in vitro or in vivo as appropriate. Thus, the DNA polymer may be prepared by the enzymatic ligation of appropriate DNA fragments, by conventional methods such as those described by D. M. Roberts et al., Biochemistry 24, 5090, 1985.

The DNA fragments may be obtained by digestion of DNA containing the required sequences of nucleotides with appropriate restriction enzymes, by chemical synthesis, by enzymatic polymerisation, or by a combination of these methods.

Digestion with restriction enzymes may be performed in an appropriate buffer at a temperature of 20°-70° C., generally in a volume of 50 ml or less with 0.1-10 mg DNA. Enzymatic polymerisation of DNA may be carried out in vitro using a DNA polymerase such as DNA polymerase 1 (Klenow fragment) in an appropriate buffer containing the nucleotide triphosphates dATP, dCTP, dGTP and dTTP as required at a temperature of 10°-37° C., generally in a volume of 50 ml or less.

Enzymatic ligation of DNA fragments may be carried out using a DNA ligase such as T4 DNA ligase in an appropriate buffer at a temperature of 4° C. to 37° C., generally in a volume of 50 ml or less.

The chemical synthesis of the DNA polymer or fragments may be carried out by conventional phosphotriester, phosphite or phosphoramidite chemistry, using solid phase techniques such as those described in ‘Chemical and Enzymatic Synthesis of Gene Fragments—A laboratory Manual’ (ed. H. G. Gassen and A. Lang), Verlag Chemie, Weinheim (1982), or in other scientific publications, for example M. J. Gait, H. W. D. Matthes M. Singh, G. S. Sproat and R. C. Titmas, Nucleic Acids Research, 1982, 10, 6243; B. S. Sproat and W. Bannwarth, Tetrahedron Letters, 1983, 24, 5771; M. D. Matteucci and M. H. Caruthers, Tetrahedron Letters, 1980, 21, 719; M. D. Matteucci and M. H. Caruthers, Journal of the American Chemical Society, 1981, 103, 3185; S. P. Adams et al., Journal of the American Chemical Society, 1983, 105, 661; N. D. Sinha, J. Biernat, J. McMannus and J. Koester, Nucleic Acids Research, 1984, 12, 4539; and H. S. D. Matthes et al., EMBO Journal, 1984, 3, 801. Preferably an automated DNA synthesiser (for example, Applied Biosystems 381A Synthesiser) is employed.

The DNA polymer is preferably prepared by ligating two or more DNA molecules which together comprise a DNA sequence encoding the polypeptide.

The DNA molecules may be obtained by the digestion with suitable restriction enzymes of vectors carrying the required coding sequences.

The precise structure of the DNA molecules and the way in which they are obtained depends upon the structure of the desired product. The design of a suitable strategy for the construction of the DNA molecule coding for the polypeptide is a routine matter for the skilled worker in the art.

In particular, consideration may be given to the codon usage of the particular host cell. The codons may be optimised for high level expression in E. coli using the principles set out in Devereux et al. (1984) Nucl. Acid Res., 12, 387.

The expression of the DNA polymer encoding the polypeptide in a recombinant host cell may be carried out by means of a replicable expression vector capable, in the host cell, of expressing the DNA polymer. Novel expression vectors also form part of the invention.

The replicable expression vector may be prepared in accordance with the invention, by cleaving a vector compatible with the host cell to provide a linear DNA segment having an intact replicon, and combining said linear segment with one or more DNA molecules which, together with said linear segment, encode the polypeptide, under ligation conditions.

The ligation of the linear segment and more than one DNA molecule may be carried out simultaneously or sequentially as desired.

Thus, the DNA polymer may be preformed or formed during the construction of the vector, as desired. The choice of vector will be determined in part by the host cell, which may be prokaryotic, such as E. coli, or eukaryotic, such as mouse C127, mouse myeloma, chinese hamster ovary, fungi e.g. filamentous fungi or unicellular ‘yeast’ or an insect cell such as Drosophila. The host cell may also be in a transgenic animal. Suitable vectors include plasmids, bacteriophages, cosmids and recombinant viruses derived from, for example, baculoviruses or vaccinia.

The DNA polymer may be assembled into vectors designed for isolation of stable transformed mammalian cell lines expressing the fragment e.g. bovine papillomavirus vectors in mouse C127 cells, or amplified vectors in chinese hamster ovary cells (DNA Cloning Vol. II D. M. Glover ed. IRL Press 1985; Kaufman, R. J. et al., Molecular and Cellular Biology 5, 1750-1759, 1985; Pavlakis G. N. and Harner, D. H. Proceedings of the National Academy of Sciences (USA) 80, 397-401, 1983; Goeddel, D. V. et al., European Patent Application No. 0093619, 1983).

The preparation of the replicable expression vector may be carried out conventionally with appropriate enzymes for restriction, polymerisation and ligation of the DNA, by procedures described in, for example, Sambrook et al., cited above. Polymerisation and ligation may be performed as describe above for the preparation of the DNA polymer. Digestion with restriction enzymes may be performed in an appropriate buffer at a temperature of 20°-70° C., generally in a volume of 50 ml or less with 0.1-10 mg DNA.

The recombinant host cell is prepared, in accordance with the invention, by transforming a host cell with a replicable expression vector of the invention under transforming condition. Suitable transforming conditions are conventional and are described in, for example, Sambrook et al., cited above, or “DNA Cloning” Vol. II, D. M. Blover ed., IRL Press Ltd, 1985.

The choice of transforming conditions is determined by the host cell. Thus, a bacterial host such as E. coli, may be treated with a solution of CaCl2 (Cohen et al., Proc. Nat. Acad. Sci., 1973, 69, 2110) or with a solution comprising a mixture of RbCl, MnCl2, potassium acetate and glycerol, and then with 3-[N-morpholino]-propane-sulphonic acid, RbCl and blycerol or by electroporation as for example descried by Bio-Rad Laboratories, Richmond, Calif., USA, manufacturers of an electroporator. Mammalian cells in culture may be transformed by calcium co-precipitation of the vector DNA onto the cells or by using cationic liposimes. The invention also extends to a host cell transformed with a replicable expression vector of the invention.

Culturing the transformed host cell under conditions permitting expression of the DNA polymer is carried out conventionally, as described in, for example, Sambrook et al., and “DNA Cloning” cited above. Thus, preferably the cell is supplied with nutrient and cultured at a temperature below 45° C.

The protein product is recovered by conventional methods according to the host cell. Thus, where the host cell is bacterial such as E. coli and the protein is expressed intracellularly, it may be lysed physically, chemically or enzymatically and the protein product isolated from the resulting lysate. Where the host cell is mammalian, the product is usually isolated from the nutrient medium.

Where the host cell is bacterial, such as E. coli, the product obtained from the culture may require folding for optimum functional activity. This is most likely if the protein is expressed as inclusion bodies. There are a number of aspects of the isolation and folding process that are regarded as important. In particular, the polypeptide is preferably partially purified before folding, in order to minimise formation of aggregates with contaminating proteins and minimise misfolding of the polypeptide. Thus, the removal of contaminating E. coli proteins by specifically isolating the inclusion bodies and the subsequent additional purification prior to folding are important aspects of the procedure.

The folding process is carried out in such a way as to minimise aggregation of intermediate-folded states of the polypeptide. Thus, careful consideration needs to be given to, among others, the salt type and concentration, temperature, protein concentration, redox buffer concentrations and duration of folding. The exact condition for any given polypeptide generally cannot be predicted and must be determined by experiment.

There are numerous methods available for the folding of proteins from inclusion bodies and these are known to the skilled worker in this field. The methods generally involve breaking all the disulphide bonds in the inclusion body, for example with 50 mM 2-mercaptoethanol, in the presence of a high concentration of denaturant such as 8M urea or 6M guanidine hydrochloride. The next step is to remove these agents to allow folding of the proteins to occur. Formation of the disulphide bridges requires an oxidising environment and this may be provided in a number of ways, for example by air, or by incorporating a suitable redox system, for example a mixture of reduced and oxidised glutathione.

Preferably, the inclusion body is solubilised using 8M urea, in the presence of mercaptoethanol, and protein is folded, after initial removal of contaminating proteins, by addition of cold buffer. Suitable buffers may be identified using the techniques described in L Doll et al, ‘Perspectives in Protein Engineering and Complementary Technologies’, Mayflower Publications, 66-69, 1995. A suitable buffer for many of the SCR constructs described herein is 20 mM ethanolamine containing 1 mM reduced glutathione and 0.5 mM oxidised glutathione. The folding is preferably carried out at a temperature in the range 1 to 5oC over a period of 1 to 4 days.

If any precipitation of aggregation is observed, the aggregated protein can be removed in a number of ways, for example by centrifugation or by treatment with precipitants such as ammonium sulphate. Where either of these procedures are adopted, monomeric polypeptide is the major soluble product.

If the bacterial cell secretes the protein, folding is not usually necessary.

The polypeptide portion of the derivative of the invention may include a C-terminal cysteine to facilitate post translational modification. A soluble polypeptide including a C-terminal cysteine also forms part of the invention. Expression in a bacterial system is preferred for proteins of moderate size (up to ˜70 kDa) and with <˜8 disulphide bridges. More complex proteins for which a free terminal Cys could cause refolding or stability problems may require stable expression in mammalian cell lines (especially CHO). This will also be needed if a carbohydrate membrane binding element is to be introduces post-translationally. The use of insect cells infected with recombinant baculovirus encoding the polypeptide portion is also a useful general method for preparing more complex proteins and will be preferred when it is desired to carry out certain post-translational processes (such as palmitoylation) biosynthetically (see for example, J. J. Page et al J. Biol. Chem. 264, 19147-19154, 1989)

A preferred method of handling proteins C-terminally derivatised with cysteine is as a mixed disulphide with mercaptoethanol or glutathione or as the 2-nitro, 5-carboxyphenyl thioderivative as generally described below in Methods.

Peptide membrane binding elements may be prepared using standard solid state synthesis such as the Merrifield method and this method can be adapted to incorporate required non-peptide membrane binding elements such as N-acyl groups derived from myristic or palmitic acids at the N terminus of the peptide. In addition activation of an amino acid residue for subsequent linkage to a protein can be achieved during chemical synthesis of such membrane binding elements. Examples of such activations include formation of the mixed 2-pyridyl disulphide with a cyteine thiol or incorporation of an N-haloacetyl group. Peptides can optionally be prepared as the C-terminal amide.

This invention also provides for alternative methods of linking CD59 to a peptidic membrane binding elements as described in WO98/02454.

After the linkage reaction, the polypeptide conjugate can be isolated by a number of chromatographic procedures described in WO98/02454. The conjugate may be characterized by a number of techniques including high performance gel filtration, SDS polyacrylamide gel electrophoresis, isoelectric focussing, or mass spectrometry.

The compounds described by this invention are preferably administered as pharmaceutical compositions.

Accordingly, the present invention also provides a pharmaceutical composition comprising a derivative of the invention in combination with a pharmaceutically acceptable carrier.

Therapeutic compositions according to the invention may be formulated in accordance with routine procedures for administration by any route, such as oral, topical, parenteral, sublingual or transdermal or by inhalation. The compositions may be in the form of tables, capsules, powders, granules, lozenges, creams or liquid preparations, such as oral or sterile parenteral solutions or suspensions or in the form of a spray, aerosol or other conventional method for inhalation.

The topical formulations of the present invention may be presented as, for instance, ointments, creams or lotions, eye ointments and eye or ear drops, impregnated dressings and aerosols, and may contain appropriate conventional additives such as preservatives, solvents to assist drug penetration and emollients in ointments and creams.

The formulations may also contain compatible conventional carriers, such as cream or ointment bases and ethanol or oleyl alcohol for lotions. Such carriers may be present as from about 1% up to about 98% of the formulation. More usually they will form up to about 80% of the formulation.

Tablets and capsules for oral administration may be in unit dose presentation form, and may contain conventional excipients such as binding agents, for example syrup, acacia, gelatin, sorbitol, tragacanth, or polyvinlypyrollidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium laurylsulphate. Tablets may also contain agents for the stablisation of polypeptide drugs against proteolysis and absorption-enhancing agents for macromolecules. The tablets may be coated according to methods well known in normal pharmaceutical practice.

Suppositories will contain conventional suppository bases, e.g. cocoa-butter or other glyceride.

For parenteral administration, fluid unit dosage forms are prepared utilizing the compound and a sterile vehicle, water being preferred. The compound, depending on the vehicle and concentration used, is dissolved in the vehicle. In preparing solutions the compound can be dissolved in water for injection and filter sterilised before filling into a suitable vial or ampoule and sealing.

Parenteral formulations may include sustained-release systems such as encapsulation within microspheres of biodegradable polymers such as poly-lactic co-clycolic acid.

Advantageously, agents such as a local anesthetic, preservative and buffering agents can be dissolved in the vehicle. To enhance the stability, the composition can be frozen after filling into the vial and the water removed under vacuum. The dry lyophilised powder is then sealed in the vial and an accompanying vial of water for injection may be supplied to reconsitute the liquid prior to use. Advantageously, a surfactant or wetting agent is included in the composition to facilitate uniform distribution of the compound.

Compositions of this invention may also suitably be presented for administration to the respiratory tract as a snuff or an aerosol or solution for nebulizer, or as a microfine powder for insufflation, alone or in combination with an inert carrier such as lactose. In such a case the particles of active compound suitably have diameters of less than 50 microns, preferably less than 10 microns for example diameters in the range of 1-50 microns, 1-10 microns or 1-5 microns. Where appropriate, small amounts of anti-asthmatics and bronchodilators, for example sypathomimetic amines such as isoprenaline, isotharine, salbutamol, phenylephrine and ephedrine; xanthine derivatives such as theophylline and aminophyllne and corticosteroids such as prednisolone and adrenal stimulants such as ACTH may be included.

Microfine powder formulations may suitably be administered in an aerosol as a metered dose or by means of a suitable breath-activated device.

Suitable metered dose aerosol formulations comprise conventional propellants, cosolvents, such as ethanol, surfactants such as oleyl alcohol, lubricants such as oleyl alcohol, desiccants such as calcium sulphate and density modifiers such as sodium chloride.

Suitable solutions for a nebulizer are isotonic sterilised solutions, optionally buffered, at for example between pH 4-7, containing up to 20 mg ml-1 of compound but more generally 0.1 to 10 mg ml-1, for use with standard nebulisation equipment.

The quantity of material administered will depend upon the potency of the derivative and the nature of the complaint be decided according to the circumstances by the physician supervising treatment. However, in general, an effective amount of the polypeptide for the treatment of a disease or disorder is in the dose range of 0.01-100 mg/kg per day, preferably 0.1 mg-10 mg/kg per day, administered in up to five doses or by infusion.

No adverse toxicological effects are indicated with the compounds of the invention within the above described dosage range.

The invention also provides a derivative of the invention for use as a medicament.

The inventor further provides a method of treatment of disorders amenable to treatment by a soluble peptide fragment of CD59, DAF or other therapeutic agent which comprises administering a soluble derivative of said soluble peptide according to the invention, and the use of a derivative of the invention for the preparation of a medicant for treatment of such disorders.

In one preferred aspect, the present invention relates to the use of human CD59 or DAF derivatives in the therapy of disorders involving complement activity and various inflammatory and immune disorders including, but not limited to, those listed below.

Disease and Disorders Involving Complement Neurological Disorders

multiple sclerosis
stroke
Guillain Barr\acute{be} Syndrome
traumatic brain injury
Parkinson's disease
allergic encephalitis
Alzheimer's disease
   Disorder of Inappropriate or Undesirable Complement Activation
haemodialysis complications
hyperacute allograft rejection
xenograft rejection
corneal graft rejection
interleukin-2 induced toxicity during IL-2 therapy
paroxysmal nocturnal haemoglobinuria
   Inflammatory Disorders
Ulcerative Colitis
Crohn's Disease
adult respiratory distress syndrome
thermal injury including burns or frostbite
uveitis
psoriasis
asthma
acute pancreatitis
Scleroderma
   Post-Ischemic Reperfusion Conditions
myocardial infarction
balloon angioplasty
atherosclersis (chloesterol-induced & restenosis
hypertension
post-pump syndrome in cardiopulmonary bypass or renal haemodialysis
renal ischaemia
intestinal ischaemia
   Infectious Diseases or Sepsis
multiple organ failure
septic shock
   Immune Complex Disorders and Autoimmune Diseases
rheumatiod arthritis
systemic lupus erthematosus (SLE)
dermatolmyositis
SLE nephritis
proliferative nephritis
Kawasaki's disease
glomerulonephritis
haemolytic anemia
myasthenia gravis
   Reproductive Disorders
antibody- or complement-mediated infertility
   Wound Healing

In the above methods, the subject is preferably a human.

The following Methods and Examples illustrate the invention.

### GENERAL METHODS USED IN EXAMPLES

(i) DNA Cleavage

Cleavage of DNA by restriction endonucleases was carried out according to the manufacturer's instructions using supplied buffers. Double digests were carried out subcutaneously if the buffer conditions were suitable for both enzymes. Otherwise double digests were carried out sequentially where the enzyme requiring the lowest salt condition was added first to the digest. Once the digest was complete the salt concentration was altered and the second enzyme added.

(ii) DNA Ligation

Ligations were carried out using T4 DNA ligase purchased from Promega, as described in Sambrook et al, (1989) Molecular Cloning: A Laboratory Manual 2nd Edition, Cold Spring Harbour Laboratory Press.

(iii) Plasmid Isolation

Plasmid isolation was carried out by the alkaline lysis method described in Sambrook et al, (1989) Molecular Cloning: A Laboratory Manual 2nd Edition. Cold Spring Harbour Laboratory Press or by one of two commercially available kits: the Promega Wizard™ Plus Minipreps or Qiagen Plasmid Maxi kit according to the manufacturer's instructions.

(iv) DNA Fragment Isolation

DNA fragments were excised from agarose gels and DNA extracted using one of three commercially available kits: the QIAEX gel extraction kit or Qiaquick gel extraction kit (QIAGEN Inc., USA), or GeneClean (Bio 101 Inc, USA) according to the manufacturer's instructions.

(v) Introduction of DNA into E. coli

Plasmids were tranformed into E. coli XL1-Blue (Stratagene), HMS174(DE3) (Novagen, UK) or UT5600(DE3) (see below) that had been made competent using calcium chloride as described in Sambrook et al, (op. cit.). UT5600 was purchased from New England Biolabs (#801-I) and was converted to a DE3 lysogen by Dr A. Topping, Zeneca Life Science Molecules, Billingham UK. UT5600 was isolated as a mutant of K12 strain RW193 (itself derived from AB1515) which was insensitive to colicin-B (McIntosh et al. (1979) J. Bact. 137 p653). It was not initially known that ompT had been lost, but further work by the same gropu showed that protein a (now OmpT) was lacking (Earhart et al (1979) FEMS Micro Letts 6 p277). The nature of the mutation was determined to be a large deletion (Elish et al (1988) J Gen Micro 134 1355.

(vi) DNA Sequencing

DNA sequencing was contracted out to Lark (Saffron Walden, Essex UK) or MWG (Milton Keynes, UK).

(vii) Production of Oligonucleotides

Oligonucleotides were purchased from Cruachem (UK) or Genosys-Sigma (Pampisford, Coambridgeshire UK).

(viii) Polymerase Chain Reaction Amplification of DNA

Purified DNA or DNA fragments from ligation reactions or DNA fragments excised and purified from agarose gels were amplified by PCR from two primers complementary to the 5′ ends of the DNA fragment. Approximately 0.1-10 microg of DNA was mixed with commercially available buffers for PCR amplification such as 10 mM Tris pH 8.3 (at 25oC), 50 mM KCl, 0.1% gelatin; MgCl2 concentrations were varied from 1.5 mM to 6 mM to find a suitable concentration for each reaction. Oligonucleotide primers were added to a final concentration of 2 microM; each dNTP was added to a final concentration of 0.2 mM. 1 unit of Taq DNA polymerase was then added to the reaction mixture (purchased from a commercial source, e.g. Gibco). The final reaction volume varied from the 20 microL to 100 microL, which was overlayed with mineral oil to prevent evaporation. Thermal cycling was then started on a thermal cycler such as the PCR machine from MJ Research. A typical example of conditions used was 94oC for 5 minute, 55oC for 1 minute, and 72 oC for 2 minutes; however, the optimal temperatures for cycling can be determined empirically by workers skilled in the art. The DNA fragment was amplified by repeating this temperature cycle for a number of times, typically 30 times.

(ix) Colorimetric Determination of Protein Concentration

Protein concentration determination utilised a colorometric method utilising Coomassie Plus Protein Assay Reagent (Pierce Chemical Company) according to the manufacturer's instructions. The assay used an APT154 reference standard prepared using similar methodology to that described in WO 98/02454, Example 6.

(x) Sodium Dodecyl Sulphate Polyacrylamide Gel Electrophoresis (SDS PAGE)

SDS PAGE was carried out generally using the Novex system (Invitrogen) according to the manufacturer's instructions. Prepacked gels of 4-20% acrylamide were usually used. Samples for electrophoresis, including protein molecular weight standards (for example LMW Kit, Pharmacia or Novex Mark 12) were usually diluted in 1% (w/v) SDS-containing buffer (with or without 5% (v/v) 2-mercaptoethanol), and left at room temperature for about 10 to 30 min before application to the gel.

(xi) Identification of CD59 by Western Blot

For certain procedures, it is necessary to characterise the expression of recombinant CD59 by an immunological method termed a Western blot. In this method, proteins to be analysed are separated by SDS-PAGE, transferred to a protein binding membrane such as polyvinylidene difluoride (PVDF), and then probed with an antibody that is specific for the target protein. Typically, the binding of the first antibody is detected by the addition of an enzyme-labelled secondary antibody and an appropriate solution which contains a chromogenic substrate. One procedure for the transfer of proteins to a protein-binding membrane was as follows. After SDS-PAGE, the proteins on the gel were transferred by electrotransfer to a protein-binding surface such PVDF. In this procedure, two sheets of filter paper (3M, Whatman) soaked in 0.3M Tris, 10% (v/v) methanol, pH10.4, were placed on the anode of an electroblotter (Semi-dry blotter, Biorad). These filter papers were then overlayed by a further two sheets of filter paper soaked in 25 mM Tris, 10% (v/v) methanol, pH10.4. The SDS-PAGE gel was then placed on the top of the PVDF membrane, and overlayed with two sheets of filter paper soaked in 25 mM Tris, 192 mM 6-amino-n-caproic acid, 10% (v/v) methanol. The cathode of the electroblotter was then placed on top of the stack of filter papers, gel and membrane, and the proteins transferred by passing a current between the electrodes at 15V for 30 minutes. Subsequent steps for the detection of the transferred proteins were described in the Novex WesternBreeze System (Invitrogen). For the detection of human CD59, a rat anti-Cd59 monoclonal antibody YTH53.1 (Davies et al., J. Exp. Med. 170, 637, 1989) was used together with an enzyme-labeled anti-rat secondary antibody.

(xii) Reduction of Disulphides and Modification of Thiols in Proteins

There are a number of methods used for achieving the title goals. The reasons it may be necessary to carry out selective reduction of disulphides is that during the isolation and purification of multi-thiol proteins, in particular during refolding of fully denatured multi-thiol proteins, inappropriate disulphide pairing can occur. In addition, even if correct disulphide paring does occur, it is possible that a free cysteine in the protein may become blocked, for example with glutathione or cysteine. These derivatives are generally quite stable. In order to make them more reactive, for example for subsequent conjugation to another functional group, they need to be selectively reduced, with for example dithiothreitol (DTT) or Tris (2-carboxyethyl) phosphine. HCl (TCEP) then optionally modified with a function which is moderately unstable. An example of the latter is Ellmans reagent (DTNB) which gives a mixed disulphide. In the case where treatment with DTNB is omitted, careful attention to experimental design is necessary to ensure that dimerisation of the free thiol-containing protein is minimised. Reference to the term ‘selectively reduced’ above means that reaction conditions eg. duration, temperature, molar ratios of reactants have to be carefully controlled so that reduction of disulphide bridges within the natural architecture of the protein is minimised. All the reagents are commercially available eg. from Sigma or Pierce.

The following general examples illustrate the type of conditions that may be used and that are useful for the generation of free thios and their optional modification. The specific reaction conditions to achieve optimal thiol reduction and/or modification are ideally determined for each protein batch.

TCEP may be prepared as a 20 mM solution in 50 mM Hepes (approx. pH 4.5) and may be stored at −40 degrees C. DTT may be prepared at 10 mM in sodium phosphate pH 7.0 and may be stored at −40 degrees C. All of the above reagents are typically used at molar equivalence or molar excess, the precise concentrations ideally identified experimentally. The duration and the temperature of the reaction are similarly determined experimentally. Generally the duration would be in the range 1 to 24 hours and the temperature would be in the range 2 to 30 degrees C. Excess reagent may be conveniently removed by buffer exchange, for example using Sephadex G25. A suitable buffer is 0.1M sodium phosphate pH7.0.

Purifications of CD59 from Human Urine

Urine was collected into 10 mM azide/5 mM benzamidine over approximately 48 hrs. the urine was then passed through a fluted coarse filter to remove aggregates and then concentrated to approximately 150 mls in a Pellican concentrator fitted with a membrane cassette with a 10 kDa MW cut-off membrane. Insoluble material was removed by centrifugation a 10,0000×g for 30 minutes. The supernatant was then applied to a CNBr-activated Sepharose 4B affinity column prepared with the rat monoclonal anti-CD59 antibody YTH 53.1 (Davies et al., J. Exp. Med. 170, 637, 1989). the column was washed overnight with 1M NaCl and bound material eluted with 4M MgCl₂. The protein content of each 1 ml fraction eluted from the column was determined by measuring absorbance at OD280 nm. The fractions containing the most protein were then pooled and dialysed through a 10 kDa MW cut off membrane into a solution containing 0.9% NaCl, and then dialysed by a similar procedure into PBS. The dialysed protein was then concentrated using a stirred cell ultrafiltration device (Amicon) fitted with a 10 kDa MW cut-off membrane. The material may be further purified by gel filtration in 10 mM Hepes, 140 mM NaCl, pH7.4, on a Superdex S-75 fast protein liquid chromatography system (Pharmacia) or Sephadex G-75. This method gave a yield of around 7 mg pure protein from 20 L urine.

Expression and Purification of Recombinant Soluble CD59 from CHO Cells

Soluble CD59 as expressed in a recombinant form from Chinese Hamster Ovary cells as follows. Briefly, the polymerase chain reaction was used to produce a truncated cDNA encoding soluble CD59 from a full length cDNA (Davies et al. J. Exp. Med. 170, 637, 1989). A mutation was introduced into the cDNA at codon 18 of the mature protein which changed the Asn codon for Ala. The procedure for this site-directed mutagenesis can be performed by a number of methods including the Quickchange mutagenesis kit (Stratagene). To introduce the modified gene into the CHO expression plasmid pDR2EF1alpha, the polymerase chain reaction was used with two oligonucleotides; the first oligonucleotide was complementary to the first seven codons at the N-terminus of the mature Cd59 protein; and the 3′ oligonucleotide introduces a termination codon immediately following the codon for Asn-70 of the CD59 cDNA. These oligonucleotides were also designed to contain recognition sequences for restriction endonucleases compatible with the polylinker site of the CHO expression vector. The DNA fragment resulting from the PCR amplification was ligated into a CHO expression vector and this plasmid tranfected with calcium phosphate into CHO cells. Cells that had become stabily transfected were selected from untransfected cells by growth in medium that contained the antibiotic hygromycin. Individual tranformants were picked and for each clone the expression of CD59 was analysed by ELISA. The highest expressing clone was chosen for large-scale production of CD59 using a variety of techniques including the use of cell factories (Nunc).

To purify the CD9, the culture medium was precleared by centrifugation at 10,000×g for 30 minutes. The soluble CD59 was then purified using an immunoaffinity column containing the monoclonal antibody YTH53.1 (Davies et al. J. Exp. Med. 170, 637, 1989), as described above. The protein was then stored in PBS at concentrations up to 5 mg/mL at −70 oC. Soluble CD59 was purified from culture supernatants after expression in recombinant baculovirus, Pichia pastoris or CHO cells. Briefly, culture medium that contains the soluble CD59 was precleared by centrifugation at 10,000×g for 30 minutes. The soluble CD59 was then purified as described in (xiii) above using an antibody affinity column prepared with the monoclonal antibody YTH53.1 and followed by gel filtration.

Preparation of C56 Euglobulin

C56 euglobulin was an essential reagent that was used for the C5b6-initiated reactive lysis of erythrocytes. C56 euglobulin was generated in and purified from some acute-phase sera from post-trauma individuals (such as sports injuries, surgery or childbirth). Blood was drawn from donors in the acute phase of inflammation and allowed to clot at room temperature. To each 10 nls of serum, 0.5 mls of yeast suspension was added and the mixture incubated overnight on a rotator at room temperature. The serum was centrifuged to remove the yeast and dialysed against 0.02M Na/K phosphate, pH 5.4. The precipitate (containing the C56 euglobulin) was collected by centrifugation and redisolved in 0.01M Na/K phosphate/0.05M NaCl, pH7.0 containing 25% v/v glycerol.

C5b6-Initiated Reactive Lysis of Erythrocytes

Guinea pig erythrocytes (TCS Microbiological, UK) were washed twice in PBS and resuspended to 5% by volume in PBS/0.05% CHAPS. 50 microL of these cells were placed in the wells of a round-bottomed microtitre plate. Samples to be tested were diluted in PBS/0.05% CHAPS and 50 microL of these test solutions then added to the wells containing the guinea pig erythrcytes. The plate was then incubated at 37 degrees Celsius for 20 minutes to allow binding of the samples to the erythrocytes. The microtitre plates were then centrifuged at 1000 rpm for 5 minutes to pellet the cells using a benchtop centrifuge. The supernatants were removed and the cell pellets resuspended in 50 microL PBS/10 mM EDTA. These cell suspensions were then incubated with 10 microL of C56 euglobulin solution (between 1:50 to 1:500 dilution) in PBS/10 mM EDTA. This solution was mixed with the cells by placing the microtiter plate on a microtitre plate shaker for 2 minutes. To this solution was then added 90 microL of a dilution of normal human serum (from 1:50 to 1:500 in PBS/10 mM EDTA). The solutions were mixed by placing the microtitre plate on a plate shaker for a further 2 minutes. The plate was then incubated at 37 degrees Celsius for 30 minutes. To determine the degree haemolysis, the plate was then places in a benchtop centrifuge and spun at 1800 rpm for 3 minutes. 100 microL of the supernatant was transferred to a clear flat bottomed microtitre plate and the absorbance at 412 nm measured spectroscopically. As controls, guinea pig erthrocytes were treated in an identical manner to the test samples with the following exceptions. In the first stage of the assay, the control samples were incubated with 50 microL of PBS/20 mM EDTA for 20 minutes at 37 degrees Celsius. After centrifugation, a spontaneous lysis control was prepared by resuspending the cells in 150 microL PBS/10 mM EDTA; by contrast, for the maximum bysis control, the cells were resuspended in 150 microL water.

Fluorescent Labeling of Proteins

Two antibodies were labelled with fluorophores to provide two fluorescent probes that react with cellular components that localise at the cell surface in lipid rafts. The rat monoclonal anti-CD59 antibody YTH 53.1 (0.75 mg; Davies et al., J. Exp. Med. 170, 637, 1989) in PBS was labelled using the Alexa 546 Protein Labeling Kit (Molecular Probes Inc, Oregon, USA) according to the manufacturer's instructions. The final molar ratio of Alexa 546 to antibody was 3.4:1. A rabbit polyclonal antibody that binds to the B subunit of cholera toxin (1 mg; Biogenesis, Dorset, UK) according to the manufacturer's instructions. The final molar ratio of Cy2 to antibody was 2.2:1.

To detect APT070 on the cell surface 1 mg of a mouse monoclonal antibody, 3e10, raised against the first three short consensus repeat domains of human CR1 Labeling Kit (Amersham Pharmacia Biotech, Little Chalfont, UK) according to the manufacturer's instructions. The final molar ratio of Cy3 to antibody was approximately 6:1.

Fluorescence Microscopy

Fluorescence microscopy was used to charaterise the distribution of various cellular components that localise at the cell surface in lipid rafts. The components that were chosen were the ganglioside GM₁, a major constituent of lipid rafts, and the GPI-anchored protein CD59. GM₁ was detected using the B subunit of cholera toxin conjugated to FITC (FITC-CTB; Sigma-Aldrich Chemical Co., Gillingham, UK) and a fluorescently labelled cholera toxin B-subunit antibody described in (xvii). Endogenous CD59 was detected with the fluorescently labelled antibody YTH53.1 described in (xvii) above.

15 ml of Raji cells were grown in suspension culture were harvested and washed twice in PBS and finally resuspended in 1 ml of PBS. The cells were counted on a haemocytometer. For all incubations non-stick microtubes were used (anachem, Luton, UK). A total of 501 cells was used and contained 5×10⁵ Raji cells. These cells were then incubated with a variety of compounds (See Example 13 for specific incubation details). After fixation, 10 microlitres of cells were mounted on a glass slide and a coverslip were sealed with clear nail varnish. Samples were viewed using the following filters; for green fluorescence a FITC filter, excitation wavelength 465-495 nm; and for red fluorescence, a G2A filter, excitation wavelength 510-560 nm.

Non-specific interactions between the Cy-3 labeled 3e10 antibody and the Cy-2 labeled anti-CTB were not detected. Furthermore, for each fluorescent marker used, there was no crossover between the red and green fluorescence using the FITC and G2A filter.

Confocal Microscopy

For confocal microscopy, cells were treated with a variety of compounds and antibodies as described above. The cells were fixed and mounted as described and visualised using an Axiophot microscope (Carl Zeiss) coupled to a Colour Coolview CCD colour camera. Filter settings for the simultaneous detection of red and green fluorescence were used. The digital images were processed using Photoshop software (Adobe Systems). Cell images were obtained that visualised either the surface of the cell or cell sections. These latter images revealed the localisation of compounds intracellularly.

### EXAMPLE 1

Synthesis and Characterization of a Lipid-Raft Targeted Derivative of Soluble Human Urinary CD59 (APT632)

### EXAMPLE 2

Synthesis and Characterization of a Lipid-Raft Targeted Derivative of Human Recombinant Soluble CD59 (APT637)

### EXAMPLE 3

An Alternative Method for the Production of Urinary (APT2047) and Recombinant (APT2059) human CD59 Lipid-Raft Targeted Derivatives Using Linkage Through Protein Carbohydrate

### EXAMPLE 4

A Method for the Preparation of Recombinant Human CD59 with a C-Terminal Cysteine, Expressed in Yeast (APT633)

### EXAMPLE 5

A Method for the Preparation of Recombinant Human CD59 with a C-Terminal Cysteine, Expressed in E. coli (APT635)

### EXAMPLE 6

A Method for the Preparation of Recombinant Human CD59 with a C-Terminal Cysteine, Expressed in Bacculovirus/Insect Cells (APT2060)

### EXAMPLE 7

A Method for the Preparation of Recombinant Human CD59 with a C-Terminal Cysteine, Expressed in Chinese Hamster Ovary Cells (APT2061)

### EXAMPLE 8

A Method for the Conjugation of the Membrane-Localising Agent APT542 to APT633, APT635, APT2060 or APT2061

### EXAMPLE 9

A Method for the Synthesis of the Free Cysteine Form of a Membrane-Localising Agent (APT544)

### EXAMPLE 10

A Method for the Synthesis of the Lipid-Raft Targeted Fluorescent Probe APT2087

### EXAMPLE 11

A Method for the Synthesis of the Lipid-Raft Targeted Fluorescent Probe APT2104

### EXAMPLE 12

A Method for the Synthesis of the Lipid-Raft Targeted Fluorescent Probe APT2105

### EXAMPLE 13

Demonstration by Fluorescence Microscopy of Colocalisation of Proteins Modified with Membrane-Targeting Peptides and Known Lipid Raft Markers

### EXAMPLE 14

Demonstration by Confocal Microscopy of the Intracellular Localization of Lipid Raft-Targeted Compounds

### EXAMPLE 15

A Method for the Synthesis and Characterization of APT530 (SEQ ID No: 10)

### EXAMPLE 16

A Method for the Synthesis and Characterization of APT2334 (SEQ ID No: 11)

### EXAMPLE 17

Demonstration of Internalisation of APT070 in Cultured Cells.

### EXAMPLE 18

Demonstration of Lysosomal Localisation of APT2104 in Cultured Cells.

### EXAMPLE 1

Synthesis and Characterization of a Lipid-Raft Targeted Derivative of Soluble Human Urinary CD59 (APT632)

APT632 was synthesized in two steps from soluble CD59 isolated from human urine (u-hCD59; Seq. ID No. 1) as described in Methods. u-hCD59 in PBS (200 μL of a 1.9 mg/mL solution) was mixed with 2-iminothiolane, and a solution of tris-2-carboxyethyl phosphine (4 μL of a 10 mM solution in 10 mM Hepes, pH7.4) added, and the mixture left overnight at room temperature. To this solution, 10 μL of APT542 (21 mM in dimethyl sulphoxide; Seq. ID No. 2) was added and incubated at room temperature for 2 h. The product APT632 was characterized by the appearance of a protein species which migrated at approximately 21 kDa as analysed by SDS-PAGE as described in methods. A reactive lysis assay (described in Methods) demonstrated that APT632 protected guinea pig erthrocytes from complement-mediated lysis by human serum at a concentration greater than 0.5 nM. The activity of APT632 was similar to the potency of the GPI-anchored form of CD59 that had been extracted from human erthrocytes.

### EXAMPLE 2

Synthesis and Characterization of a Lipid-Raft Targeted Derivative of Human Recombinant Soluble CD59 (APT637)

APT637 was sythesized in two steps from soluble human CD59 that was expressed in a recombinant form from chinese hamster ovary cells (APT634; Seq. ID No.3). APT 634 in PBS (200 μL of a 300 μM solution was mixed with 2-iminothiolane (6 μL of a 10 mM solution) and the mixture incubated at room temperature for 30 minutes. The solution was then dialysed into PBS to remove unreacted 2-iminothiolane, and a solution of tris-2-carboxyethyl phosphine (4 μL of a 10 mM solution in 10 mM Hepes, pH7.4) added, and the mixture left overnight at room temperature. To this solution, 10 μL of APT542 (21 mM in dimethyl sulphoxide) was added and incubated at room temperature for 2 h. The product APT637 was characterized by the appearance of a protein species which migrated at approximately 10 kDa as analysed by SDS-PAGE as described in methods. A reactive lysis assay (described in Methods) demonstrated that APT637 protected guinea pig erthrocytes from complement-mediated lysis by human serum at a concentration greater than 0.5 nM. The activity of APT632 was similar to the potency of the GPI-anchored form of CD59 that had been extracted from human erthrocytes.

### EXAMPLE 3

An Alternative Method for the Production of Urinary (APT2047) and Recombinant (APT2059) human CD59 Lipid-Raft Targeted Derivatives Using Linkage Through Protein Carbohydrate

APT2047 is a conjugate of APT634 (Seq. ID No. 3) and APT542 (Seq. ID No.2), and APT2059 is a conjugate of APT631 (Seq. ID No.1) and APT542, in which the linkage of each pair of compounds is through a modified carbohydrate moiety on the CD59 protein. APT2047 and APT2059 are synthesized in three steps from APT634 or APT631 at a concentration of 1 mg/ml with 10 mM sodium periodate for 1 h in the dark, in a solution of 0.1M sodium acetate, pH5.5 to remove excess sodium periodate and glycerol. In the second step, the sodium periodate-treated proteins are reacted with a solution of (4-[4-N-maleimidophenyl]butyric acid hydrazide hydrochloride (MPBH) at a final concentration of 1 mg/ml for 2 h at room temperature to generate APT2047 and APT2059. The synthesis of these proteins is confirmed by the appearance of a novel proteinaceous species that migrates at approximately 10 kDa or 20 kDa by SDS-PAGE under non-reducing conditions, respectively. In addition, these proteins protect guinea pig erthrocytes from complement-mediated lysis by human serum at a concentration greater than 0.5 nM.

### EXAMPLE 4

A Method for the Preparation of Recombinant Human CD59 with a C-Terminal Cysteine, Expressed in Yeast (APT633)

APT633 is a protein that comprises soluble human CD59 and a C-terminal cysteine residue following position 81 of mature CD59 protein. The protein was expressed in a recombinant form in Pichia pastoris cells. The polymerase chain reaction was used to produce a truncated cDNA encoding soluble CD59 from a full length cDNA (Davies et al. J. Exp. Med. 170, 637, 1989). The 5′ oligonucleotide was complementary to 20 bases of the first 7 codons at the N-terminus of the mature CD59 protein, and the 3′ oligonucleotide introduced a cysteine codon and a termination codon immediately following the codon for Ser-81 of the mature CD59 protein. These oligonucleotides were also designed to contain recognition sequences for restriction endonucleases XhoI and EcoRI which are compatible with the polylinker site of the vector pUCPIC (a derivative of pUC19 that contains the alpha-factor leader sequence and multiple cloning site from pPIC9K (Invitogen). The DNA fragment resulting from the PCR amplification was then ligated into pUCPIC DNA and transformed into the XL1-Blue strain of E. coli (Stratagene). The transfected cells are selected by growth on a petri dish containing LB medium (Sigma) supplemented with ampicillin at a concentration of 100 micrograms/ml (LBAMP). The DNA that encodes the alpha factor and CD59 was then subcloned into the vector pPIC9K that had been digested with the restriction endonuclease BamHI and EcoRI. Purified DNA from the resulting plasmid was linearised with the restriction endonuclease PmeI for transformation into P. pastoris strain GS115 (Invitrogen) by spheroplasting according to the manufacturer's instructions. After preliminary selection for clones that are capable of growth on a minimal RD medium (1M sorbitol, 2% w/v dextrose, 1.34% yeast nitrogen base, 4×10⁻⁵% biotin, 0.005% amino acids) lacking histidine. Clones having undergone multiple integration events were then selected by resistance to the antibiotic geneticin sulphate (G418). Clones that were capable of growth in medium containing G418 at a concentration of 2 mg/mL were screened for expression of CD59. Individual colonies were inoculated in 10 mL BMG medium (100 mM potassium phosphate, pH6.0, 13.4 mg/mL yeast nitrogen base, 0.4 mg/L biotin, 1% (w/v) glycerol) and grown at 30° C. with shaking until clones reached an optical density of 6 as measured spectroscopically at a wavelength of 600 nm. The cultures were then transferred to BMM medium (100 mM potassium phosphate, pH6.0, 13.4 g/L yeast nitrogen base, 0.4 mg/L biotin, 0.5% methanol) and grown for 48 h at 30° C. with shaking. Culture supernatants were then analysed by SDS-PAGE and Western blot for the presence of APT633 which was observed as a novel proteinaceous species which migrated at approximately 8000 Da.

### EXAMPLE 5

A Method for the Preparation of Recombinant Human CD59 with a C-Terminal Cysteine, Expressed in E. coli (APT635;Seq ID. No.5)

APT635 is a protein that comprises soluble human CD59 and a C-terminal cysteine residue following codon 81 of the mature CD59 protein (Seq. ID No.5). The protein is expressed in a recombinant form in E. coli cells. The polymerase chain reaction was used to produce a truncated cDNA encoding soluble CD59 from a full length cDNA (Davies et al. J. Exp. Med. 170, 637, 1989). The 5′ oligonucleotide was complementary to 20 bases of the first 7 codons at the N-terminus of the mature CD59 protein, and the 3′ oligonucleotide introduced a cysteine codon and a termination codon immediately following the codon for the Ser-81 of the mature CD59 protein. These oligonucleotides were also designed to contain recognition sequences for restriction endonucleases compatible with the polylinker site of pBROC413 (described in WO 94/00571). The DNA fragment resulting from the PCR amlplification was then ligated into pBROC413 DNA and transformed into the UT5600(DE3) cells transfected by DNA encoding APT635 was then grown with shaking overnight at 37° C. In LBAMP. This overnight culture was then diluted 1:100 in LBAMP medium and grown with shaking at 37° C. until the culture reached an optical density of 1.0 as determined by absorbance at a wavelength of 600 nm. To this culture was added a solution of isopropyl beta-D-thiogalactopyranoside to a final concentration of 1 mM. The culture was then grown for a further 3 hours with shaking at 37° C. The cells are harvested by centrifugation and inclusion bodies isolated as described in WO 94/00571. The expression of APT635 was determined by SDS-PAGE and confirmed by the appearance of a novel protein species that migrates at approximately 8000 Da.

### EXAMPLE 6

A Method for the Preparation of Recombinant Human CD59 with a C-Terminal Cysteine, Expressed in Bacculovirus/Insect Cells (APT2060; Seq ID. No. 4) APT2060 is a protein that comprises soluble human CD59 and a C-terminal cysteine residue following codon 81 of the mature CD59 protein (Seq. ID No.4) The protein was expressed in a recombinant form in a baculovirus expression system. The polymerase chain reaction was used to produce a truncated cDNA encoding soluble CD59 from a full length cDNA (Davies et al. J. Exp. Med. 170, 637, 1989). The 5′ oligonucleotide was complementary to 20 bases of the first 7 codons at the N-terminus of the mature CD59 protein, and the 3′ oligonucleotide introduced a cysteine codon and a termination codon immediately following the codon for Ser-81 of the mature CD59 protein. These oligonucleotides were also designed to contain recognition sequences for restriction endonucleases compatible with the polylinker site of pBacPAK 8 baculovirus transfer vector (Clontech). The DNA fragment resulting from the PCR amplification was then ligated into pBacPAK 8 DNA. This plasmid was then transfected into Sf9 cells with Bacfectin (Clontech) and BacPAK6 viral DNA which had been cut with the restriction endonuclease Bsu36I. This mixture was deposited onto a 50% confluent monolayer of Sf9 cells and left at 28° C. for 3 days. The supernatant was removed and a plaque assay performed on serial dilutions of the transfection supernatant as described in Baculovirus Expression Protocols, Methods in Molecular Biology series, ed. C. Richardson). Individual plaques were then picked into 0.5 mL IPL-41 medium (Gibco BRL) containing 1% foetal calf serum. The mixture was left at room temperature for 15 minutes and 100 microL of this solution used to inoculate a 50% confluent monolayer of Sf9 cells. The cells were then left to become infected for 4-5 days at 28° C. After this time, the supernatant was removed and assayed for CD59 expression by Western blot as described in methods. For scale-up of the recombinant virus, the supernatant was used as an inoculum to infect more Sf9 cell monolayers as described above; alternatively, the supernatant can be used to infect Sf9 cells grown in suspension cultures. In this method, 100 mL Sf9 cells at a concentration of 5×10⁶ cells/ml in IP-41 medium containing 1% FCS were inoculated with 50 microL of viral supernatant. The culture was shaken for 507 days at 27° C. and cells removed by centrifugation. The recombinant virus may be stored at 4° C. until use. APT2060 may be detected by Western blot as described in Methods and purified using an affinity column as described.

### EXAMPLE 7

A Method for the Preparation of Recombinant Human CD59 with a C-Terminal Cysteine, Expressed in Chinese Hamster Ovary Cells (APT2061; Seq. ID. No. 6)

APT2061 is a protein that comprises soluble human CD59 and a C-terminal cysteine residue at position 71 of the mature protein. The protein may be expressed in a recombinant form in chinese hamster ovary cells as described in Methods. Briefly, the polymerase chain reaction is used to produce a truncated cDNA encoding soluble CD59 from a full length cDNA (Davies et al. J. Exp. Med. 170, 637, 1989). The 5′ oligonucleotide introduces a cysteine codon and a termination codon immediately following the codon for Asn-70 of the CD59 cDNA. These oligonucleotides can also designed to contain recognition sequences for restriction endonucleases compatible with the polylinker site of a CHO expression vector, as described.

### EXAMPLE 8

A Method for the Conjugation of the Membrane-Localising Agent APT542 to APT633, APT635, APT2060 or APT2061 to Generate Compounds APT2062 (Seq. ID No.7), APT2063 (Seq. ID No.8), APT2064 (Seq. ID No.7) and APT2065 (Seq. ID No.9).

Compounds APT2062, APT2063, APT2064 and APT2065 are generated by treating their parent compounds APT633, APT635, APT2060 and APT2061 with a single molar equivalent of tris-2-carboxyethyl phosphine (TCEP; in 10 mM Hepes, pH7.4) overnight at room temperature. To this mixture is added a solution containing 5 molar equivalents of APT542 for 2 h at room temperature. APT542 was synthesized and characterized as described in WO 98/02454. (Example 2)

APT 2063 was synthesized according to the method described. The mass of APT2063 was determined as 11482 Da which correlated with the expected mass of 11496 Da.

### EXAMPLE 9

A Method for the Synthesis of the Free Cysteine Form of a Membrane-Localising Agent: Preparation of N-Myristoyl-Gly-Ser-Ser-Lys-Ser-Pro-Ser-Lys-Lys-Lys-Lys-Lys-Lys-Pro-Gly-Glu-Cys-NH₂ (APT544)

APT542 (250 μL of a 21.6 mM solution in DMSO) And TCEP (400 μL of a 100 mM solution in water) were mixed. Dethiopyridylation was monitored by HPLC (10-90% acetonitrile in 0.1% TFA) and evidenced by the disappearance of APT542 at 13.9 mins and the appearance of APT544 at 14.2 mins. After 2 h the reaction mixture was purified by preparative HPLC. The product-containing fractions were taken to low volume on the rotary evaporator and APT544 obtained as a white solid after lyophilisation. Treatment of an aqueous solution of APT544 with 1 mM DTT yielded no increase in absorbance at 343 nm indicating complete removal of the thiopyridyl function.

### EXAMPLE 10

A Method for the Synthesis of the Lipid-Raft Targeted Fluorescent Probe (1): Preparation of N-Myristoyl-Gly-Ser-Ser-Lys-Ser-Pro-Ser-Lys-Lys-Lys-Lys-Lys-Lys-Pro-Gly-Glu-Cys-(S-5-Succinimido Fluorescein)-NH₂ (APT2087)

APT544 (3.0 mg, 1.50 μmol) was dissolved in degassed 20 mM sodium phosphate, 150 mM NaCl, pH 7.2, 1 mM EDTA (500 μL) and fluorescein-5-maleimide (4.65 μmol, 2 mg in 100 μL DMF) added in one portion. The mixture was stirred at 4° C. in the dark overnight. Reaction completion was monitored by HPLC, evidenced by disappearance of APT 544 at 12.8 mins and the appearance APT 2087 at 14.5 mins. The reaction was purified and lyophilised as before to yield a yellow solid.

### EXAMPLE 11

A Method for the Synthesis of the Lipid-Raft Targeted Fluorescent Probe (2): Synthesis of N-Myristoyl-Gly-Ser-Ser-Lys-Ser-Pro-Ser-Lys-Lys-Lys-Lys-Lys-Lys-Pro-Gly-Glu-Cys(S-Succinimido Alexafluor™488 C₅)-NH₂ (APT2104)

APT544 (2.40 mg, 1.20 μmol) was dissolved in degassed 20 mM sodium phosphate, 150 mM NaCl, pH 7.2, 1 mM EDTA (500 μL). Alexafluor™488 C₅ maleimide (1 mg, 1.39 μmol, Molecular Probes Inc, Oregon, USA) was dissolved in DMSO (30 μL) and added in one portion to the APT544 solution. The mixture was stirred at 4° C. in the dark overnight, purified and lyophilised as before to yield the title compound as an orange solid. The retention time was identical to that of APT544 (14.2 mins) but unlike APT 544, APT2104 absorbed strongly at 343 nm.

### EXAMPLE 12

A Method for the Synthesis of the Lipid-Raft Targeted Fluorescent Probe (3): Preparation of N-Myristoyl-Gly-Ser-Ser-Lys-Ser-Pro-Ser-Lys-Lys-Lys-Lys-Lys-Lys-Pro-Gly-Glu-Cys(S-Succinimido Alexafluor™546 C₅)-NH₂ (APT2105)

APT544 (2.40 mg, 1.20 μmol) Was dissolved in degassed 20 mM sodium phosphate, 150 mM NaCl, pH 7.2, 1 mM EDTA (500 μL). Alexafluor™546 C₅ maleimide (1 mg, 1.03 μmol, source as above) was dissolved in DMSO (20 μL) and added in one portion to the APT544 solution. The mixture was stirred at 4° C. in the dark overnight, purified and lyophilised as before to yield the title compound as a purple solid. RT 15.7 mins; MALDI Mass Spec: C₁₃₁H₂₀₅Cl₃N₂₉O₃₆S₄ requires 2997.83 Da; Observed 3000 Da.

### EXAMPLE 13

Demonstration by Fluorescence Microscopy of Colocalisation of Proteins Modified with Membrane-Targeting Peptides and Known Lipid Raft Markers

Commonly used markers of lipid rafts are cholera toxin B subunit (CTB) that binds specifically to the ganglioside GM₁, itself a major constituent of lipid rafts; and CD59, which was used in this study as an example of a GPI anchored protein. These raft-associated markers were found to co-localise with three different compounds: the derivatized fluorophores APT2104 and APT2105; and APT070, a derivative of a protein that is not normally associated with lipid rafts.

(i) Endogenous CD59 and APT2104:

Endogenous CD59 was detected with the monoclonal antibody YTH53.1 that had been labeled with Alexa Fluor 546 as described in (xvii, herein). 2 microlitre of labeled-YTH53.1 antibody (7 micromolar in PBS) and 2 micorlitre of APT2104 (22 micromolar in PBS) were added to Raji cells as described in (xviii) and incubated for 60 min and 37 C. The cells were washed three times with 0.5 ml ice-cold PBS and finally resuspended in 50 microlitre of PBS. The cells were examined by fluorescence microscopy as described in section xviii.

(ii) Cholera Toxin B Subunit and APT2105

2 microlitre of FITC-CTB (100 micromolar in PBS), and 2 microlitre of APT2015 (20 micromolar in PBS) were added to the Raji cells as described in (xviii) and incubated for 60 min at 37 C. The cells were washed three times with 0.5 ml of ice-cold PBS. To enhance the weak fluorescence signal from of the cholera toxin, the cells were resuspended in 48 microlitre of PBS and 2 microlitre of Cy2-anti cholera toxin B subunit antibody (6 micromolar; described in xvii above) and incubated for 60 min at 37 C. The cells were washed three times with 0.5 ml of ice-cold PBS. The cells were then fixed in 50 microlitre of 4%(w/v) paraformaldeyde, 0.125%(v/v) glutaraldehyde for 10 min at 22 C. The cells were washed with 0.5 ml ice-cold PBS and finally resuspended in 50 microlitre of PBS. The cells were examined by fluorescence microscopy as described in section xviii.

(iii) APT070 and Cholera Toxin B Subunit

2 microlitre of FITC-CTB (100 micromolar in PBS) and 0.5 microlitre of APT070 (100 micromolar in PBS; described in WO 98/02454; [SCR1-3]-Cys-S-S-[MSWP-1]; example 8) were added to the cell suspension and incubated for 60 min at 37 C. The cells were washed three times with 0.5 ml of ice-cold PBS. To enhance the weak fluorescence signal from of the cholera toxin and to detect the APT070 on the cell surface, the cells were resuspended in 46 microlitre of PBS and 2 microlitre of both Cy2-anti cholera toxin B subunit antibody 96 micromolar) and Cy3-labelled 3e10 was added and incubated for 60 min at 37 C. The cells were washed three times with 0.5 ml of ice-cold PBS. The cells were fixed in 50 microlitre of 4%(w/v) parafomrmaldehyde, 0.125%(v/v) glutaraldehyde for 10 min at 22 C. The cells were washed with 0.5 ml ice-cold PBS and finally resuspended in 50 microlitre of PBS. The cells were examined by fluorescence microscopy as described in section xviii.

In the above experiments a discrete punctuate pattern was seen in each; of APT2105 with FITC-CTB and of endogenous CD59 with APT2104 and also APT070 with FITC-CTB on Raji cells. These patterns showed a very similar distribution of fluorescence between the targeted proteins and the markers for lipid rafts. This provides strong evidence that modification with the membrane targeting peptide. APT542 confers selective binding to lipid rafts within the cell membrane.

### EXAMPLE 14

Demonstration by Confocal Microscopy of the Intracellular Localization of Lipid Raft-Targeted Compounds

This example follows the same procedure as described in example 13 with the following modifications. In certain experiments, lipid raft targeted compounds were added to the cells either singly, or in combination with other compounds. The compounds were then visualized with fluorescent antibodies or in the case of APT2104 and APT2105 by their intrinsic fluorescence, in each case using a confocal microscope as described in methods. In the following cases intracellular fluorescence was seen deriving from the lipid raft targeting peptides: Endogenous CD59 and APT2104; FITC-CTB and APT2105; APT070 and FITC-CTB; APT2104; APT2105. These data demonstrate that the derivatisation of compounds with peptides that localize the compound to a lipid raft also deliver the compound intracellularly.

### EXAMPLE 15

A Method for the Synthesis and Characterization of APT530 (SEQ ID No: 10)

APT530 is a protein that comprises the short consensus repeats 1,2,3 and 4 of human CD55 (decay accelerating factor, DAF), with a carboxyl terminal cysteine residue expressed in a recombinant form in E. coli cells. cDNA that encoded human DAF mRNA was generated from total brain RNA as described in Example 9A plasmid to encode APT530 was generated by PCR using the pUC-CAF plasmid as template. Primers were designed to amplify the region of the DAF gene encoding amino acids 35-285 (SCR1-4). The 5′ primer incorporated an NdeI restriction enzyme site, and a codon specifying glutamine, thereby introducing an amino terminal methionine-glutamine amino acid pair. The 3′ primer added a carboxyl terminal cysteine residue and incorporated an EcoRI restriction enzyme site. The PCR product was cloned into the pUC57/T T-vector as described, sequenced, the insert excised with NdeI and EcoRI, and ligated into pET266 (Novagen, Madison, USA). The product of this ligation is the plasmid pET99-01, which expresses DAF (SCR1-4). pET99-01 DNA was introduced into E. coli HAMS113 (see methods) and expression of the recombinant protein induced as described in Example 1The expression of APT530 was analysed by SDS-PAGE (described in methods). APT530 appeared as a unique protein product of approximately 28000 Da as estimated by comparative mobility with molecular weight standards and had a mass of 28133 Da (predicted 28148 Da) as determined by MALDI mass spectometry. Cells containing APT530 were harvested by centrifugation and inclusion bodies isolated as follows. Briefly, the cells were resuspended in lysis buffer (50 mM Tris, 1 mM ethylene diamine tetra-acetic acid (DTDA), 50 mM NaCl, pH 8.0) at 50 ml per litre of initial culture. The suspension was lysed by two passages through an Emulsiflex homogensier (Glen-Creston, Middlesex UK), followed by centrifugation at 15,000×g to purify inclusion bodies. Inclusion bodies were initially resuspended to approximately 1 mg.ml⁻¹ (as estimated from SDS-PAGE) in 100 mM Tris, 1 mM EDTA, 25 mM DTT, pH8, and subsequently diluted to a final concentration of 8M urea by the addition of 10 M urea 100 mM Tris, 1 mM EDTA, 25 mM DTT, pH8. This suspension was stirred at 4 C for 2 hours, and acidified by dialysis into 6M Urea, 10M HCl. The APT530 was refolded by rapid dilution into 20 mM ethanolamine, 1 mM EDTA, pH 11 buffer and static incubation at 4 C for 24 hours. Insoluble material was removed by centrifugation (10,000×g, 10 minutes), and soluble material buffer exchanged into Dulbecco's A PBS, pH 7.4 using an XK50×23 cm Sephadex G25 column. Refolded APT2058 was analysed by SDS-PAGE, Western blot and the effectiveness of the protein in a haemolytic assay (described in methods). Using this assay (at 1:400 dilution of human serum), the concentration of APT530 required to bring about 50% inhibition of lysis (IH₅₀) was approximately 40 nM.

### EXAMPLE 16

A Method for the Synthesis and Characterization of APT2334 (SEQ ID No: 11)

Compound APT2334 was generated by treating the parent compound APT530 (at approximately 100 μM) with a three-fold molar excess of 10 mM tris-2-carboxyethyl phosphine (TCEP: in 50 mM Hepes, pH 4.5) overnight at room temperature. To this mixture was added a solution containing five molar equivalents of MSWP-1 (Example 2 of WO 98/02454) in 100% DMSO for 2 hours at room temperature. APT2334 was characterized by observation of a mobility shift on non-reducing SDS-PAGE of approximately 2000 Da, consistent with the addition of a single molecule of APT542 to APT530 and has a molecular mass of 30125 Da (predicted 30148 Da). The compound was assayed in the haemolytic assay (at 1:400 dilution of human serum) and an IH₅₀ value 0.2 nM was found.

### EXAMPLE 17

Demonstration of Internalisation of APT070 in Cultured Cells.

Porcine aortic endothelial (PAE) cells were grown to confluence on poly-D-lysine-coated coverslips. Cells were washed once with HEPES-buffered Ham's F12, and were subsequently kept in this medium. APT070 (1 μM) was added and the cells were incubated for 30 min at 37° C. Cells were washed three times with PBS prior to fixation with paraformaldehyde (3.75% (w/v) in 200 mM HEPES-KOH, pH 7.2) for 20 min. Cells were then washed three times 95 min each) with HEPES-buffered Ham's F12. In order to distinguish internalised APT070 from outer membrane-associated compound, some of the cells were then permeabilised by incubation for 10 min with 0.1% (v/v) Triton X-100 in PBS. These cells were then washed three times with PBS. All of the samples were incubated with blocking buffer (0.25% (w/v) type B gelatin in PBS) for 15 min. Cell-associated APT070 was immunodetected using monoclonal antibody 3e10 which had been labelled with the fluorophore AlexaFluor-488 (Molecular Probes Inc.). The antibody was diluted 1/100 in blocking buffer and incubated with the samples for 30 min at room temperature. Samples were washed three times (5 min each) with blocking buffer prior to mounting and imaging by fluorescence microscopy.

### EXAMPLE 18

Demonstration of Lysosomal Localisation of APT2104 in Cultured Cells.

COS-7 cells were grown to confluence on poly-D-lysine-coated coverslips. Cells were washed once with HEPES-buffered DMEM, and were subsequently kept in this medium. APT2104 was added at concentrations in the range 0.1-1.0 μM and the cells were incubated for different times at 37° C. For lysosomal staining, 75 nM ‘Lysotracker Red’ (Molecular Probes Inc.) was added to the cells for the final hour of the incubation. Cells were then washed three times DMEM and either viewed live in a cavity slide by fluorescence microscopy or else fixed with paraformaldehyde (as described above) prior to mounting and imaging by fluorescence microscopy.
Chemical Structures

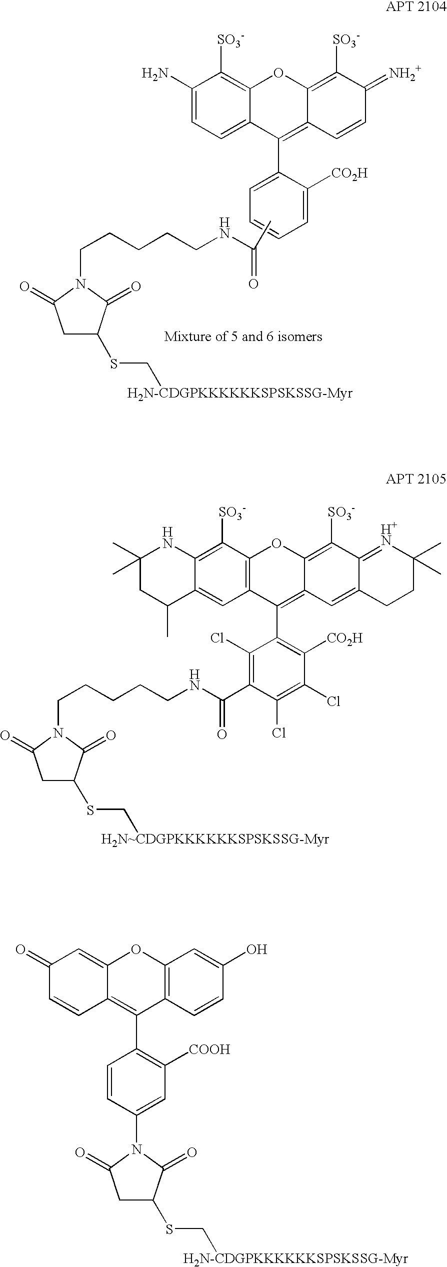

1. A compound comprising a membrane binding peptide, wherein the peptide is modified by the addition of a C-terminal cysteine residue, wherein the C-terminal cysteine residue is covalently associated with a fluorescein derivative which is covalently associated through a S-5-succinimido group, and wherein the compound is selected from the group consisting of:
a) N-Myristoyl-Gly-Ser-Ser-Lys-Ser-Pro-Ser-Lys-Lys-Lys-Lys-Lys-Lys-Pro-Gly-Glu-Cys(S-5-succinimido fluorescein)-NH₂ (APT2087) (SEQ ID NO: 13),
b) N-Myristoyl-Gly-Ser-Ser-Lys-Ser-Pro-Ser-Lys-Lys-Lys-Lys-Lys-Lys-Pro-Gly-Glu-Cys(S-5-succinimido Alexafluor™488 C₅)-NH₂ (APT2104) (SEQ ID NO: 14), and
c) N-Myristoyl-Gly-Ser-Ser-Lys-Ser-Pro-Ser-Lys-Lys-Lys-Lys-Lys-Lys-Pro-Gly-Glu-Cys(S-5-succinimido Alexafluor™546 C₅)-NH₂ (APT2105) (SEQ ID NO: 15).

2. The compound of claim 1 interacts selectively with components of lipid rafts.

3. The compound according to claim 2, in which the components of lipid rafts include one or more of phosphatidylserine, phosphatidyl glycerol, glycosphingolipids, cholesterol, GPI-anchored proteins associated with lipid rafts, and other protein components of lipid rafts that may be found normally on the exoplasmic face of the cell.

4. The compound of claim 1 modulates the function of the lipid rafts either to affect intracellular signaling or to change extracellular functions mediated through the raft domains.

5. The compound of claim 1 mediates internalization of the peptide.

6. The compound of claim 1 wherein the peptide is a soluble complement regulatory molecule, including but not restricted to CD59 and DAF, which is targeted to lipid rafts and the signaling pathways that are associated with lipid rafts.

7. The compound according to claim 6 wherein the soluble complement regulatory molecule is a modified CD59 or DAF peptide, which is targeted to lipid rafts.

8. The compound according to claim 7, wherein the modified CD59 or DAF peptide is selected from the group consisting of:
APT635 (Seq ID No. 5),
APT2063 (Seq ID No. 8),
APT530 (Seq ID No. 10 ),
APT2334 (Seq ID No. 11),
APT070, and
APT154.

9. The compound of claim 1 further comprises a derivatised antibody, or antibody fragment which can provide a surrogate receptor localized at a lipid raft to divert a mediator interacting with a lipid raft receptor or which can neutralise a further component of a raft such as a cofator required for signaling.

10. The compound of claim 1 further comprises a derivated chemical or biological entity that possesses the physical property of fluorescence which enables lipid rafts to be identified and/or monitored.

11. The compound of claim 1 further comprises a derivatised chemical or biological entity involved in a catalytic process either as an enzyme an enzyme substrate or an enzyme inhibitor.

12. The compound of claim 1 further comprises a derivatised chemical or biological entity that can form a covalent chemical bond with proteins, sugar groups or lipids that are localized in lipid rafts thus permitting the isolation and identification of the raft component.

13. The compound according to claim 12, wherein said entity contains photo-, chemo-, or enzyme-activated crosslinking groups.

14. A process for preparing a compound of claim 1, wherein the process comprises expressing DNA encoding the peptide portion of the compound in a recombinant host cell and recovering the product and thereafter post translationally modifying the polypeptide to chemically introduce membrane binding elements with selectivity for lipid rafts.

15. The process according to claim 14, wherein the process comprises the steps of:
i) preparing a replicable expression vector capable, in a host cell, of expressing a DNA polymer comprising a nucleotide sequence that encodes said polypeptide portion;
ii) transforming a host cell with said vector;
iii) culturing said transformed host cell under conditions permitting expression of said DNA polymer to produce said polypeptide; and
iv) recovering said polypeptide.

16-19. (canceled)

20. A pharmaceutical composition comprising a compound according to claim 1 and a pharmaceutically acceptable carrier.

21-22. (canceled)


SEQUENCE LISTING (15 sequences)

SEQ ID NO: 1 - protein, 77 aa, Homo sapiens
LQCYNCPNPTADCKTAVNCSSDFDACLITKAGLQVYNKCWKFEHCNFNDVTTRLRENELTYYCCKKDLCNFNEQLEN

SEQ ID NO: 2 - protein, 17 aa, Artificial Sequence
  Description of Artificial Sequence Synthetic peptide
  see specification as filed for detailed description of amino and carboxy terminal modifications
GSSKSPSKKKKKKPGDC

SEQ ID NO: 3 - protein, 70 aa, Artificial Sequence
  Description of Artificial Sequence Synthetic peptide
LQCYNCPNPTADCKTAVACSSDFDACLITKAGLQVYNKCWKFEHCNFNDVTTRLRENELTYYCCKKDLCN

SEQ ID NO: 4 - protein, 82 aa, Artificial Sequence
  Description of Artificial Sequence Synthetic peptide
LQCYNCPNPTADCKTAVNCSSDFDACLITKAGLQVYNKCWKFEHCNFNDVTTRLRENELTYYCCKKDLCNFNEQLENGGTSC

SEQ ID NO: 5 - protein, 83 aa, Artificial Sequence
  Description of Artificial Sequence Synthetic peptide
MLQCYNCPNPTADCKTAVNCSSDFDACLITKAGLQVYNKCWKFEHCNFNDVTTRLRENELTYYCCKKDLCNFNEQLENGGTSC

SEQ ID NO: 6 - protein, 71 aa, Artificial Sequence
  Description of Artificial Sequence Synthetic peptide
LQCYNCPNPTADCKTAVACSSDFDACLITKAGLQVYNKCWKFEHCNFNDVTTRLRENELTYYCCKKDLCNC

SEQ ID NO: 7 - protein, 99 aa, Artificial Sequence
  Description of Artificial Sequence Synthetic peptide
LQCYNCPNPTADCKTAVNCSSDFDACLITKAGLQVYNKCWKFEHCNFNDVTTRLRENELTYYCCKKDLCNFNEQLENGGTSCCDGPKKKKKKSPSKSSG

SEQ ID NO: 8 - protein, 100 aa, Artificial Sequence [sequence omitted]

SEQ ID NO: 9 - protein, 88 aa, Artificial Sequence
  Description of Artificial Sequence Synthetic peptide
LQCYNCPNPTADCKTAVACSSDFDACLITKAGLQVYNKCWKFEHCNFNDVTTRLRENELTYYCCKKDLCNCCDGPKKKKKKSPSKSSG

SEQ ID NO: 10 - protein, 254 aa, Artificial Sequence
  Description of Artificial Sequence Synthetic peptide
MQDCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGC

SEQ ID NO: 11 - protein, 271 aa, Artificial Sequence
  Description of Artificial Sequence Synthetic peptide
  see specification as filed for detailed description of amino and carboxy terminal modifications
MQDCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGCCDGPKKKKKKSPSKSSG

SEQ ID NO: 12 - protein, 17 aa, Artificial Sequence
  Description of Artificial Sequence Synthetic peptide
  see specification as filed for detailed description of amino and carboxy terminal modifications
GSSKSPSKKKKKKPGEC

SEQ ID NO: 13 - protein, 17 aa, Artificial Sequence
  Description of Artificial Sequence Synthetic peptide
  see specification as filed for detailed description of amino and carboxy terminal modifications
GSSKSPSKKKKKKPGEC

SEQ ID NO: 14 - protein, 17 aa, Artificial Sequence
  Description of Artificial Sequence Synthetic peptide
  see specification as filed for detailed description of amino and carboxy terminal modifications
GSSKSPSKKKKKKPGEC

SEQ ID NO: 15 - protein, 17 aa, Artificial Sequence
  Description of Artificial Sequence Synthetic peptide
  see specification as filed for detailed description of amino and carboxy terminal modifications
GSSKSPSKKKKKKPGEC